(19) United States
(12) Patent Application Publication
(10) Pub. No.: US 2026/0125796 A1
(43) Pub. Date: May 7, 2026

(54) Processing Device and Processing Method

(71) Applicant: Tokyo Electron Limited, Tokyo (JP)

(72) Inventor: Mikio SATO, Yamanashi (JP)

(21) Appl. No.: 19/435,138

(22) Filed: Dec. 29, 2025

Related U.S. Application Data
(63) Continuation of application No. PCT/JP2024/023692, filed on Jul. 1, 2024.

(30) Foreign Application Priority Data
Jul. 14, 2023 (JP) 2023-115710

(51) Int. Cl. C23C 16/48; B08B 9/00; B08B 15/02; G01N 21/64; G01N 33/00

(52) U.S. Cl. CPC C23C 16/48; B08B 15/02; G01N 21/64; G01N 33/0027; B08B 9/00

(57) ABSTRACT

A processing apparatus comprises a processing chamber, a gas supply source, a first excitation light source and a second excitation light source. The gas supply source is configured to supply noble gas and a processing gas into the processing chamber. The first excitation light source is configured to irradiate the processing chamber with first excitation light to excite the noble gas to a first excited state. The second excitation light source is configured to irradiate the processing chamber with second excitation light to excite the noble gas in the first excited state to a second excited state.

### CROSS-REFERENCE TO RELATED APPLICATIONS

This application is a bypass continuation application of International Application No. PCT/JP2024/023692 having an international filing date of Jul. 1, 2024 and designating the United States, the International Application being based upon and claiming the benefit of priority from Japanese Patent Application No. 2023-115710 filed on Jul. 14, 2023, the entire contents of which are incorporated herein by reference.

### TECHNICAL FIELD

The present disclosure relates to a processing apparatus and a processing method.

### BACKGROUND

Japanese Laid-open Patent Publication No. 2008-263226 discloses a configuration in which a location where first plasma is generated is provided separately from a vacuum processing chamber accommodating a sample on the upstream side of the vacuum processing chamber, metastable atoms generated in the first plasma generation location are injected into the vacuum processing chamber, and second plasma is generated in the vacuum processing chamber.

### SUMMARY

The present disclosure provides a processing apparatus and a processing method capable of uniformly generating metastable excited atoms.

A processing apparatus according to one aspect of the present disclosure comprises a processing chamber, a gas supply source configured to supply noble gas and a processing gas into the processing chamber, a first excitation light source configured to irradiate the processing chamber with first excitation light to excite the noble gas to a first excited state, and a second excitation light source configured to irradiate the processing chamber with second excitation light to excite the noble gas in the first excited state to a second excited state.

### BRIEF DESCRIPTION OF THE DRAWINGS

FIG. 1 is a schematic cross-sectional view showing an example of a configuration of a substrate processing apparatus according to a first embodiment of the present disclosure.

FIG. 2 is a diagram showing an example of transition of argon gas from a ground state to metastable excited atoms.

FIG. 3 is a diagram showing an example of combination of excitation light that allows noble gas to make transition to metastable excited atoms.

FIG. 4 is a diagram showing an example of combination of excitation light that allows noble gas to make transition to metastable excited atoms.

FIG. 5 is a diagram showing an example of combination of excitation light that allows noble gas to make transition to metastable excited atoms.

FIG. 6 is a diagram showing an example of a configuration of a second excitation light source in the first embodiment.

FIG. 7 is a diagram showing an example of a configuration of a second excitation light source in the first embodiment.

FIG. 8 is a diagram showing an example of the relationship between a distance from a lithium fluoride window, a generation density of atoms in a first excited state, and an intensity of second excitation light.

FIG. 9 is a diagram showing an example of the relationship between a distance from a lithium fluoride window, an output of a heater, and an intensity of second excitation light.

FIG. 10 is a flowchart showing an example of substrate processing in the first embodiment.

FIG. 11 is a schematic cross-sectional view showing an example of a configuration of a substrate processing apparatus in a second embodiment.

### DETAILED DESCRIPTION

Hereinafter, embodiments of a processing apparatus and a processing method of the present disclosure will be described in detail with reference to the accompanying drawings. Further, the technique of the present disclosure is not limited to the following embodiments.

Conventionally, radicals are generated using plasma, and processes are performed using the generated radicals. However, when plasma is used, ions and electrons are also generated, which results in damages due to ion bombardment or charging. Therefore, a method in which metastable excited atoms of noble gas are generated using photoexcitation, and the energy thereof is used to dissociate a processing gas to generate radicals may be considered. However, metastable excited atoms cannot be optically excited directly from the ground state and, thus, stepwise excitation using multiple light sources is required. Accordingly, when photoexcitation is used, it is expected to uniformly generate metastable excited atoms by controlling the multiple light sources.

### First Embodiment

### [Configuration of Substrate Processing Apparatus 1]

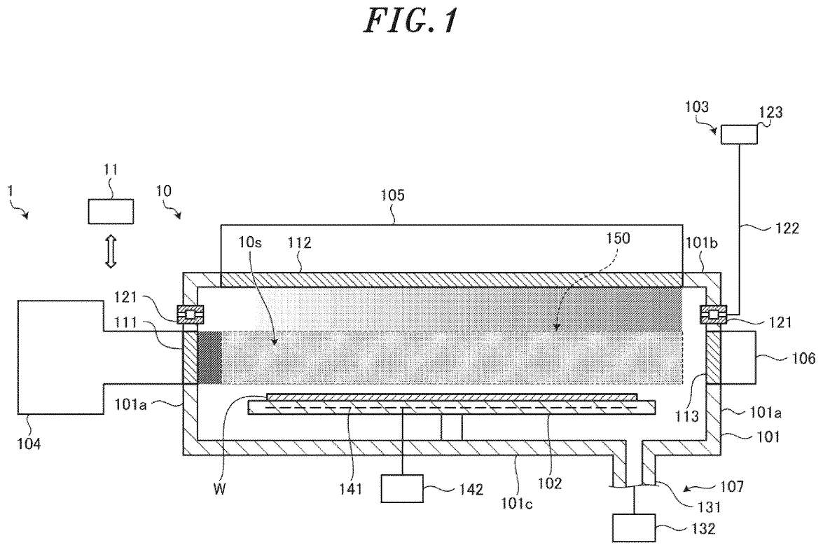

FIG. 1 is a schematic cross-sectional view showing an example of a configuration of a substrate processing apparatus according to a first embodiment of the present disclosure. As shown in FIG. 1, a substrate processing apparatus 1 includes a main body 10, and a controller 11 that controls the main body 10. The main body 10 includes a processing chamber 101, a placing table 102, a gas supply mechanism 103, a first excitation light source 104, a second excitation light source 105, an emission sensor 106, and an exhaust mechanism 107.

The processing chamber 101 is formed in a substantially cylindrical shape, and the placing table 102 is located approximately at the center of the bottom surface of the processing chamber 101. The processing chamber 101 does not necessarily have a substantially cylindrical shape, and may have any shape, such as a rectangular parallelepiped or the like. An irradiation window 111 that transmits first excitation light inputted from the first excitation light source 104, and a detection window 113 where the emission sensor 106 is provided are formed at a sidewall 101a of the processing chamber 101. The irradiation window 111 is made of, e.g., lithium fluoride. Further, an opening (not shown) through which a substrate W passes is formed at a portion (not shown) of the sidewall 101a, and the opening is opened and closed by a gate valve (not shown). Further, an irradiation window 112 that transmits second excitation light inputted from the second excitation light source 105 is formed on an upper surface 101b of the processing chamber 101. The irradiation window 112 is made of, e.g., quartz.

A substrate W to be processed is placed on the placing table 102. The placing table 102 has a substantially disc shape, and is made of ceramic such as AlN or the like. Lifting pins (not shown) for raising and lowering the substrate W are provided inside the placing table 102 to protrude and retract with respect to the upper surface of the placing table 102. The placing table 102 has a holding mechanism for a substrate W, such as an electrostatic chuck or a mechanical chuck (not shown), but the holding mechanism for a substrate W is not limited thereto.

Further, a resistance heater 141 is embedded in the placing table 102. The heater 141 heats the substrate W placed on the placing table 102 by the power supplied from a heater power supply 142. The heater 141 is divided into a plurality of regions on a substrate supporting surface of the placing table 102, and the power can be controlled for each region. Further, a thermocouple (not shown) is inserted into the placing table 102, and the power of the heater 141 is controlled for each region based on the signal from the thermocouple, thereby controlling the temperature gradient of the substrate W. The temperature gradient (gradation) of the substrate W is controlled such that the side close to the irradiation window 111 is set as a high-temperature side and the side distant from the irradiation window 111 is set as a low-temperature side between both ends of the substrate W in a diametrical direction, for example. In other words, the temperature of the placing table 102 is controlled such that the output gradation of the heater 141 is formed to correspond to the temperature gradient.

The gas supply mechanism 103 includes a shower ring 121 provided in a ring shape along the inner wall of the processing chamber 101. The shower ring 121 has a ring-shaped channel provided therein, and a plurality of injection ports that are connected to the channel and opened to the inside thereof. A gas supply part 123 is connected to the shower ring 121 through a line 122. The gas supply part 123 is provided with a plurality of gas sources and a plurality of flow rate controllers. In one embodiment, the gas supply part 123 is configured to supply a processing gas containing at least one noble gas from the corresponding gas source to the shower ring 121 via the corresponding flow rate controller. The gas supplied to the shower ring 121 is supplied into the processing chamber 101 from the plurality of injection ports. Further, the gas supply mechanism 103 is an example of the gas supply source.

The gas supply part 123 supplies a silicon-containing gas, a halogen-containing gas, a nitrogen-containing gas, noble gas, or the like, which is controlled at a predetermined flow rate, into the processing chamber 101 via the shower ring 121. In the present embodiment, the silicon-containing gas is, e.g., silane (SiH₄) gas. Further, in the present embodiment, the halogen-containing gas is, e.g., NF₃ gas. Further, in the present embodiment, the nitrogen-containing gas is, e.g., ammonia (NH₃) gas. Further, in the present embodiment, the noble gas is, e.g., argon (Ar) gas. Instead of argon gas, other noble gases such as neon (Ne) gas, krypton (Kr) gas, and xenon (Xe) gas may be used.

The first excitation light source 104 is controlled to irradiate the first excitation light, e.g., vacuum ultraviolet light having a wavelength of 106.6660 nm, to the processing chamber 101 through the irradiation window 111. In other words, the first excitation light is irradiated from the side portion of the processing chamber 101. In this case, the first excitation light is a sheet-shaped light having a width greater than or equal to the diameter of the substrate W to be processed. The sheet-shaped light may be generated using a lens and a mirror, or may be generated by laser scanning. The first excitation light source 104 may be, e.g., an excimer lamp, a dye laser, a D2 lamp, or the like. The first excitation light may be, e.g., continuous light. The first excitation light excites the noble gas supplied into the processing chamber 101 from the ground state to the first excited state. For example, the first excitation light having a wavelength of 106.6660 nm excites the argon gas supplied into the processing chamber 101 from the ground state (0 eV) to the first excited state (11.62 eV). Further, a vacuum state is maintained between the first excitation light source 104 and the irradiation window 111. In the case of using argon gas as the noble gas, the first excitation light source 104 may use vacuum ultraviolet light with a wavelength of 104.8220 nm, as the first excitation light, for example. In the case of using vacuum ultraviolet light with a wavelength of 104.8220 nm, the energy of the first excited state becomes 11.83 eV. Since the first excitation light is absorbed by the noble gas, the intensity (light amount) thereof decreases as the distance from the irradiation window 111 increases.

The second excitation light source 105 is controlled to irradiate the second excitation light, e.g., infrared light with a wavelength of 810.5921 nm, into the processing chamber 101 through the irradiation window 112. In other words, the second excitation light is irradiated from the top of the processing chamber 101. In this case, the second excitation light is a beam-shaped light that irradiates the entire substrate W to be processed. The beam-shaped light that irradiates the entire substrate W to be processed may be generated using a lens and a mirror or by laser scanning. The second excitation light may be, e.g., continuous light. The second excitation light excites the noble gas in the processing chamber 101 from the first excited state to the second excited state. For example, the second excitation light with a wavelength of 810.5921 nm excites argon gas in the processing chamber 101 from the first excited state (11.62 eV) to the second excited state (13.15 eV). In the case of using argon gas as the noble gas and vacuum ultraviolet light with a wavelength of 104.8220 nm as the first excitation light, the second excitation light may be infrared light with a wavelength of 826.6794 nm. In the case of using infrared light with a wavelength of 826.6794 nm, the energy of the second excited state becomes 13.33 eV.

Further, the second excitation light source 105 may be connected to the irradiation window 112 via an optical fiber. In this case, the second excitation light source 105 may be located at a position other than the upper portion of the processing chamber 101. In other words, the upper structure of the processing chamber 101 can be reduced. A region 150 shown in FIG. 1 is irradiated with both the first excitation light and the second excitation light, and metastable excited atoms of the noble gas are generated in the region 150. In other words, metastable excited atoms of the noble gas are generated only at the intersection of the first excitation light and the second excitation light. Further, in FIG. 1, the first excitation light and the second excitation light are indicated by shading with gradation. The region 150, where metastable excited atoms are substantially uniformly generated, is indicated by shading without gradient. Further, the wavelength combination of the first excitation light and the second excitation light and the cases of using other gases as the noble gas will be described later.

The emission sensor 106 detects the emission when the noble gas makes the transition from the second excited state to metastable excited atoms. The emission sensor 106 detects the emission in the processing chamber 101 through the detection window 113, and is controlled to output the detected emission data to the controller 11. The controller 11 measures the emission intensity distribution in the processing chamber 101 based on the inputted emission data. Further, a plurality of emission sensors 106 and a plurality of detection windows 113 may be provided. Further, the emission intensity distribution in the processing chamber 101 can be measured even when there is only one emission sensor 106 and one detection window 113 by using techniques such as optical emission tomography and integral photography. For example, when the symmetry of the shape of the processing chamber 101 is excellent, the emission intensity distribution of the entire processing space 10s in the processing chamber 101 can be measured by shifting the field of view of the emission sensor 106 at one detection window 113.

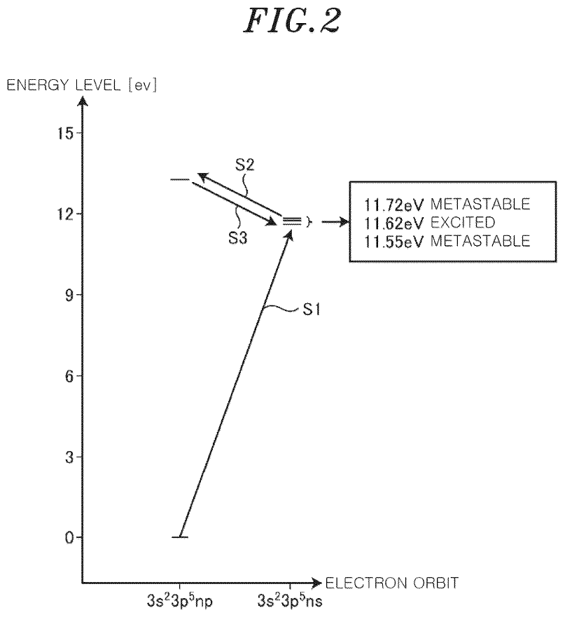

Here, the transition of noble gas to metastable excited atoms will be described with reference to FIGS. 2 to 5. FIG. 2 shows an example of the transition of argon gas from the ground state to the metastable excited atoms. As shown in FIG. 2, in the present embodiment, when argon gas in the ground state (0 eV) is irradiated with the first excitation light having a wavelength of 106.6660 nm, the argon atoms make the transition from the ground state (0 eV) to the first excited state (radiative: 11.62 eV) (step S1). Further, the electron orbit becomes 3s²3p⁶ to 3s²3p⁵4s.

When argon atoms in the first excited state are irradiated with the second excitation light having a wavelength of 810.5921 nm, the argon atoms make the transition from the first excited state to the second excited state (13.15 eV) (step S2). The electron orbit becomes 3s²3p⁵4s to 3s²3p⁵4p.

The argon atoms in the second excited state relax by spontaneously emitting light with a wavelength of 772.5886 nm or 867.0324 nm, and make the transition to metastable excited atoms of 11.55 eV or 11.72 eV (step S3). Further, they make the transition from the second excited state of 13.15 eV, to metastable excited atoms of 11.55 eV and 11.72 eV. The electron orbit becomes 3s²3p⁵4p to 3s²3p⁵4s. As described above, the metastable excited atoms of 11.55 eV and 11.72 eV cannot be optically excited directly from the ground state. In the substrate processing apparatus 1, the generation of metastable excited atoms can be detected by detecting spontaneously emitted light with wavelengths of 772.5886 nm and 867.0324 nm using the emission sensor 106.

Further, the light with wavelengths of 810.6 nm and 935.7 nm may be detected during transitions of argon atoms. Therefore, the emission sensor 106 preferably has wavelength resolution capable of distinguishing those wavelengths from the wavelengths of 772.5886 nm and 867.0324 nm. Further, when radicals in the processing gas do not emit light with wavelengths in the vicinity of 810.6 nm and 935.7 nm, the wavelengths of 810.6 nm and 935.7 nm may also be used as data indirectly indicating the generation of metastable excited atoms. In this case, the emission sensor 106 with wavelength resolution sufficient to detect wavelengths of 772.5886 nm and 867.0324 nm and wavelengths of 810.6 nm and 935.7 nm without distinguishing them may be used. Further, the emission sensor 106 capable of detecting spontaneous emission is used depending on types of noble gases.

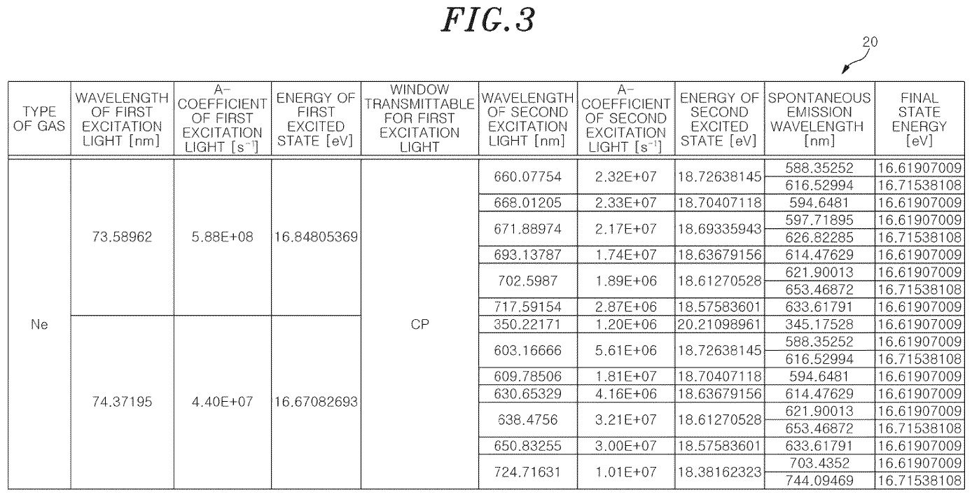

FIGS. 3 to 5 show examples of combination of excitation light in which noble gas becomes metastable excited atoms. Table 20 in FIG. 3 shows combinations of the first excitation light, the material of the irradiation window 111, the second excitation light, the spontaneous emission wavelength, and the final state energy in the case of using neon gas as noble gas. Further, the final state energy indicates the energy level of the metastable excited atoms. As shown in Table 20, in the case of neon gas, vacuum ultraviolet lights with wavelengths of 73.58962 nm and 74.37195 nm are used as the first excitation light. The A coefficients of the first excitation light are 5.88E+8(5.88×10⁸)[s⁻¹] and 4.40E+7(4.40×10⁷) [s⁻¹], respectively. Further, the A coefficients indicate the ease of emission of molecules or atoms in an excited state. The energies of the first excited state are 16.84805369 eV and 16.67082693 eV, respectively. In the case of neon gas, the wavelength of the first excitation light is short and, thus, it is difficult to use lithium fluoride as the material of the irradiation window 111. Hence, a capillary plate (CP) in which a plurality of holes with diameters of about 1 μm to several hundred μm are arranged two-dimensionally is used as the material of the irradiation window 111. The CP itself is made of a dielectric material such as lead glass. In the case of using a CP, the differential pressure is adjusted to prevent the processing gas or the like in the processing chamber 101 from leaking toward the first excitation light source 104. The energies of the first excited state are 16.84805369 eV and 16.67082693 eV, respectively.

As shown in Table 20, in the case of neon gas, when the wavelength of the first excitation light is 73.58962 nm, six wavelengths from 660.07754 nm to 717.59154 nm can be used as the wavelength of the second excitation light. The six wavelengths are 660.07754 nm, 668.01205 nm, 671.88974 nm, 693.13787 nm, 702.5987 nm, and 717.59154 nm. Table 20 also shows the A coefficient, the second excited state energy, and the spontaneous emission wavelength of each of the six wavelengths. The final state energy resulting from the transition to metastable excited atoms when each of the six wavelengths is used as the second excitation light is 16.61907009 eV or 16.71538108 eV.

Further, in the case of neon gas, when the wavelength of the first excitation light is 74.37195 nm, seven wavelengths from 350.22171 nm to 724.71631 nm can be used as the wavelength of the second excitation light. The seven wavelengths are 350.22171 nm, 603.16666 nm, 609.78506 nm, 630.65329 nm, 638.4756 nm, 650.83255 nm, and 724.71631 nm. Table 20 also shows the A coefficient, the second excited state energy, and the spontaneous emission wavelength of each of the seven wavelengths. The final state energy resulting from the transition to metastable excited atoms when each of the seven wavelengths is used as the second excitation light is 16.61907009 eV or 16.71538108 eV.

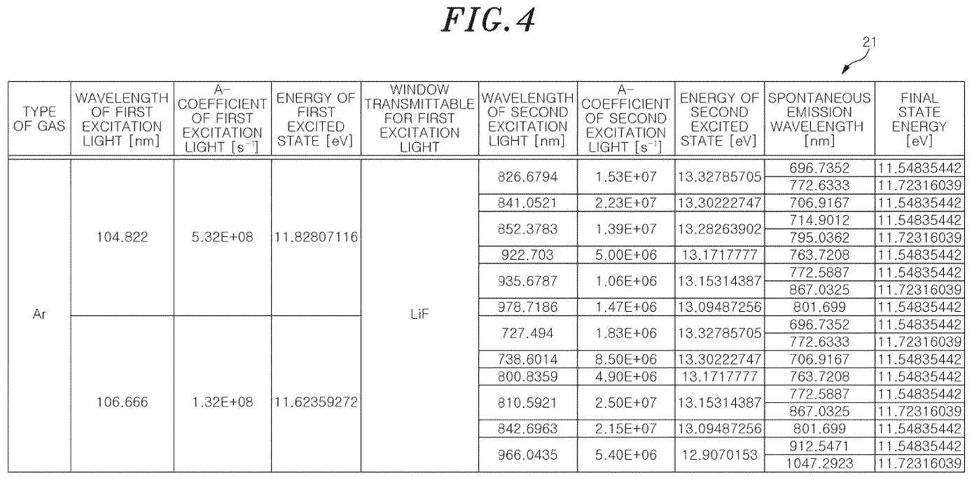

Table 21 in FIG. 4 shows combinations of the first excitation light, the material of the irradiation window 111, the second excitation light, the spontaneous emission wavelength, and the final state energy in the case of using argon gas as noble gas. As shown in Table 21, in the case of argon gas, vacuum ultraviolet light with wavelengths of 104.8220 nm and 106.6660 nm is used as the first excitation light. The A coefficients of the first excitation light are 5.32E+8(5.32×10⁸) [s⁻¹] and 1.32E+8(1.32×10⁸) [s⁻¹], respectively. Lithium fluoride (LiF) can be used as the material for the irradiation window 111.

As shown in Table 21, in the case of using argon gas with the first excitation light wavelength of 104.8220 nm, six wavelengths from 826.6794 nm to 978.7186 nm can be used as the second excitation light wavelength. The six wavelengths are 826.6794 nm, 841.0521 nm, 852.3783 nm, 922.703 nm, 935.6787 nm, and 978.7186 nm. Table 21 also lists the A coefficient, the second excited state energy, and the spontaneous emission wavelength of each of the six wavelengths. The final state energy resulting from the transition to metastable excited atoms when each of the six wavelengths is used as the second excitation light is 11.54835442 eV or 11.72316039 eV. Further, in the above explanation, the digits are omitted to indicate 11.55 eV and 11.72 eV, respectively.

Further, in the case of argon gas, when the wavelength of the first excitation light is 106.6660 nm, six wavelengths from 727.494 nm to 966.0435 nm can be used as the wavelength of the second excitation light. The six wavelengths are 727.494 nm, 738.6014 nm, 800.8359 nm, 810.5921 nm, 842.6963 nm, and 966.0435 nm. Table 21 also shows the A coefficient, the second excited state energy, and the spontaneous emission wavelength of each of the six wavelengths. The final state energy resulting from the transition to metastable excited atoms when each of the six wavelengths is used as the second excitation light is 11.54835442 eV or 11.72316039 eV.

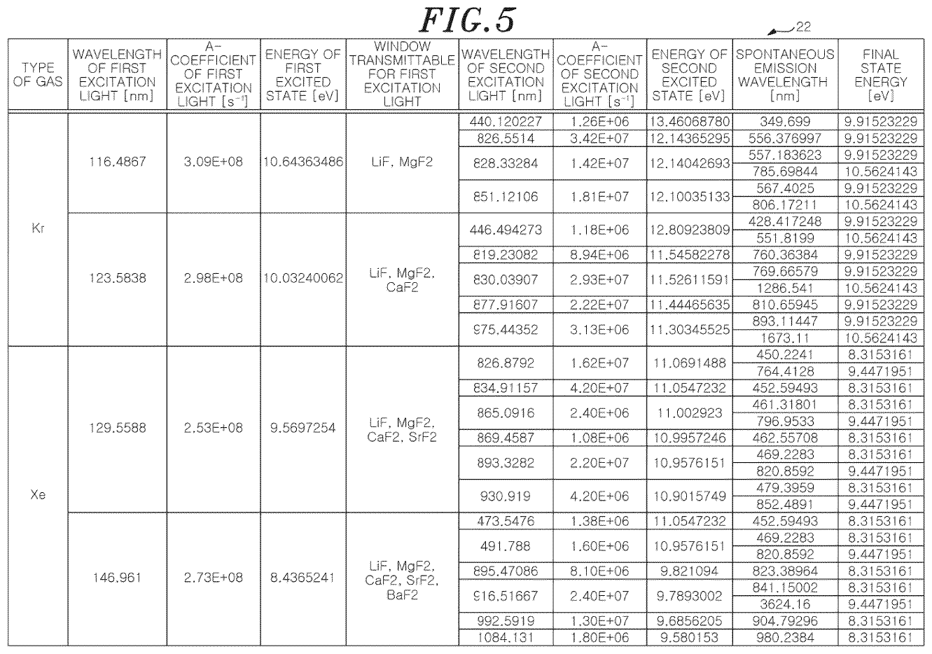

Table 22 in FIG. 5 shows combinations of the first excitation light, the material of the irradiation window 111, the second excitation light, the spontaneous emission wavelength, and the final state energy in the case of using krypton gas and xenon gas as noble gas. As shown in Table 22, in the case of krypton gas, vacuum ultraviolet light with wavelengths of 116.4867 nm and 123.5838 nm is used as the first excitation light. The A coefficients of the first excitation light are 3.09E+8(3.09×10⁸)[s⁻¹] and 2.98E+8 (2.98×10⁸)[s⁻¹], respectively. In the case of the wavelength of 116.4867 nm, lithium fluoride (LiF) and magnesium fluoride (MgF₂) can be used as the material of the irradiation window 111. In the case of the wavelength of 123.5838 nm, lithium fluoride (LiF), magnesium fluoride (MgF₂), and calcium fluoride (CaF₂) can be used as materials for the irradiation window 111.

As shown in Table 22, in the case of krypton gas, when the wavelength of the first excitation light is 116.4867 nm, four wavelengths from 440.120227 nm to 851.12106 nm can be used as the wavelength of the second excitation light. The four wavelengths are 440.120227 nm, 826.5514 nm, 828.33284 nm, and 851.12106 nm. Table 22 also shows the A coefficient, the second excited state energy, and the spontaneous emission wavelength of each of the four wavelengths. The final state energy resulting from the transition to metastable excited atoms when each of the four wavelengths is used as the second excitation light is 9.91523229 eV or 10.5624143 eV.

Further, in the case of krypton gas, when the wavelength of the first excitation light is 123.5838 nm, five wavelengths from 446.494273 nm to 975.44352 nm can be used as the wavelength of the second excitation light. The five wavelengths are 446.494273 nm, 819.23082 nm, 830.03907 nm, 877.91607 nm, and 975.44352 nm. Table 22 also shows the A coefficient, the second excited state energy, and the spontaneous emission wavelength of each of the five wavelengths. The final state energy resulting from the transition to metastable excited atoms when the five wavelengths is used as the second excitation light is 9.91523229 eV or 10.5624143 eV.

As shown in Table 22, in the case of xenon gas, vacuum ultraviolet light with wavelengths of 129.5588 nm and 146.961 nm is used as the first excitation light. The A coefficients of the first excitation light are 2.53E+8(2.53×10⁸)[s⁻¹] and 2.73E+8(2.73×10⁸)[s⁻¹], respectively. In the case of the wavelength of 129.5588 nm, lithium fluoride (LiF), magnesium fluoride (MgF₂), calcium fluoride (CaF₂), and strontium fluoride (SrF₂) can be used as the material of the irradiation window 111. In the case of the wavelength of 146.961 nm, lithium fluoride (LiF), magnesium fluoride (MgF₂), calcium fluoride (CaF₂), strontium fluoride (SrF₂), and barium fluoride (BaF₂) can be used as the material for the irradiation window 111.

As shown in Table 22, in the case of xenon gas, when the wavelength of the first excitation light is 129.5588 nm, six wavelengths from 826.8792 nm to 930.919 nm can be used as the wavelength of the second excitation light. The six wavelengths are 826.8792 nm, 834.91157 nm, 865.0916 nm, 869.4587 nm, 893.3282 nm, and 930.919 nm. Table 22 also shows the A coefficient, the second excited state energy, and the spontaneous emission wavelength of each of the six wavelengths. The final state energy resulting from the transition to metastable excited atoms when each of the six wavelengths is used as the second excitation light is 8.3153161 eV or 9.4471951 eV.

Further, in the case of xenon gas, when the wavelength of the first excitation light is 146.961 nm, six wavelengths from 473.5476 nm to 1084.131 nm can be used as the wavelength of the second excitation light. The six wavelengths are 473.5476 nm, 491.788 nm, 895.47086 nm, 916.51667 nm, 992.5919 nm, and 1084.131 nm. Table 22 also shows the A coefficient, the second excited state energy, and the spontaneous emission wavelength of each of the six wavelengths. The final state energy resulting from the transition to metastable excited atoms when each of the six wavelengths is used as the second excitation light is 8.3153161 eV or 9.4471951 eV.

Referring back to the description of FIG. 1, the exhaust mechanism 107 includes an exhaust line 131 provided at a bottom surface 101c of the processing chamber 101 and an exhaust device 132 connected to the exhaust line 131. The exhaust device 132 includes a vacuum pump, a pressure control valve, and the like. In one embodiment, the exhaust mechanism 107 is configured to adjust the pressure in the processing chamber 101 by controlling the vacuum pump and the pressure control valve.

The controller 11 includes a memory, a processor, and an I/O interface. The memory stores programs executed by the processor and recipes including process conditions. The processor executes the programs read from the memory and controls individual components of the main body 10 via the I/O interface based on the recipes stored in the memory.

For example, the controller 11 controls individual components of the substrate processing apparatus 1 to perform a substrate processing method to be described later. Specifically, for example, the controller 11 executes a step of loading and preparing a substrate W into the processing chamber 101. The controller 11 executes a step of supplying the noble gas and a processing gas into the processing chamber 101. The controller 11 executes a step of irradiating the processing chamber 101 with the first excitation light to excite the noble gas to the first excited state. The controller 11 executes a step of irradiating the processing chamber 101 with the second excitation light to allow the noble gas to make the transition from the first excited state to the second excited state. The controller 11 executes a step of dissociating the processing gas with the noble gas that has become metastable excited atoms from the second excited state, thereby processing the substrate W.

### [Detailed Description of Second Excitation Light Source 105]

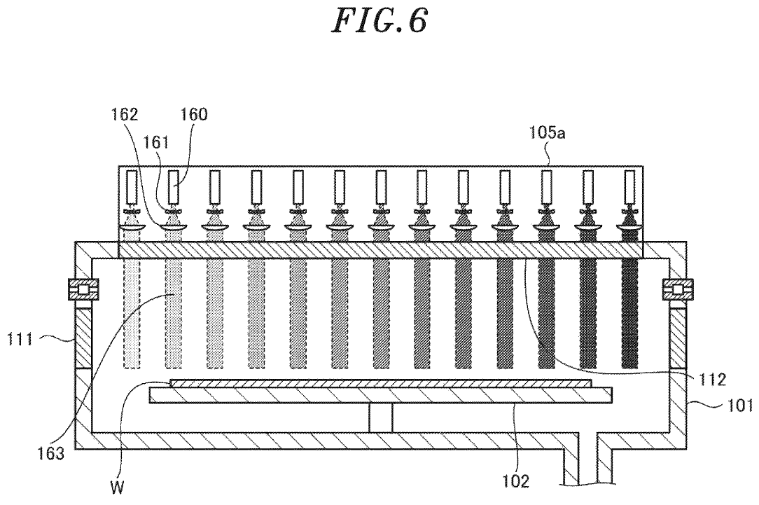

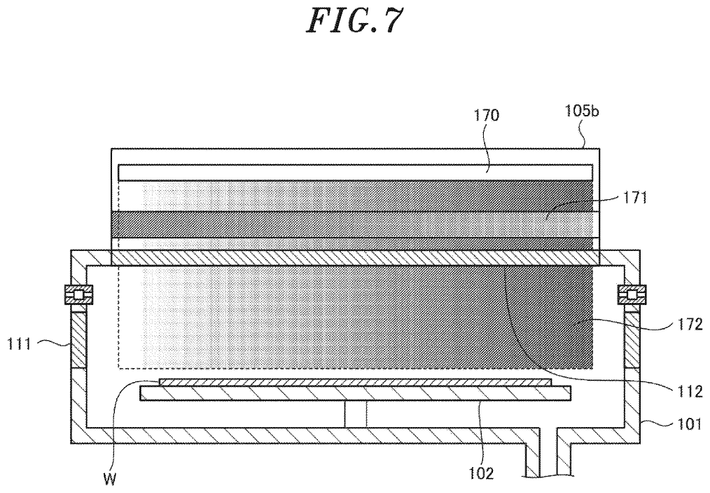

Next, the second excitation light source 105 will be described in detail with reference to FIGS. 6 and 7. FIG. 6 is a diagram showing an example of a configuration of the second excitation light source in the first embodiment. In the example shown in FIG. 6, a second excitation light source 105a is used as the second excitation light source 105. The second excitation light source 105a has multiple pairs of a light source 160 and lenses 161 and 162. The light source 160 uses, e.g., a lamp, a light emitting diode (LED), a laser, or the like, and outputs, e.g., infrared light as the second excitation light. The lens 161 is, e.g., a magnifying lens, and the lens 162 is, e.g., a collimator lens. The second excitation light outputted from the light source 160 is magnified by the lens 161 and transformed into parallel rays 163 by the lens 162, and irradiated into the processing chamber 101 through the irradiation window 112. Although FIG. 6 shows the rays 163 spaced apart from each other, there is actually no space between adjacent rays 163. Further, the second excitation light emitted from the second excitation light source 105a is controlled such that the intensity becomes weaker on the side close to the irradiation window 111 and stronger on the side distant from the irradiation window 111 in the processing chamber 101, as indicated by the gradation in FIG. 6. In other words, the second excitation light source 105a includes a plurality of light sources 160, and the intensity of the second excitation light is controlled for each of the plurality of light sources 160. In the second excitation light source 105a, the outputs of the plurality of light sources 160 are individually controlled, thereby forming the gradation in the intensity of the second excitation light from the upstream side (close to the irradiation window 111) toward the downstream side (distant from the irradiation window 111) of the first excitation light. Further, the pairs of the light source 160 and the lenses 161 and 162 may have another beam shape other than parallel rays, such as sheet light from diffusion, as long as the gradation in the intensity of the second excitation light can be formed.

FIG. 7 shows an example of a configuration of the second excitation light source in the first embodiment. In the example shown in FIG. 7, a second excitation light source 105b is used as the second excitation light source 105. The second excitation light source 105b includes a light source 170 and a light control filter 171. The light source 170 uses, e.g., a lamp, an LED, a laser, or the like and outputs, e.g., infrared light as the second excitation light. The light control filter 171 is, e.g., a liquid crystal filter, and controls the amount of light outputted from the light source 170. The second excitation light outputted from the light source 170 is controlled by the light control filter 171, and is irradiated into the processing chamber 101 through the irradiation window 112. Further, the second excitation light irradiated from the second excitation light source 105b has a low intensity on the side close to the irradiation window 111 and a high intensity on the side distant from the irradiation window 111 in the processing chamber 101, as indicated by the gradation in FIG. 7. In the second excitation light source 105b, the transmittance of the light control filter 171 is controlled to form gradation in the intensity of the second excitation light from the upstream side (close to the irradiation window 111) of the first excitation light toward the downstream side (distant from the irradiation window 111).

[Relationship Between Distance from Irradiation Window 111 and Intensity of Second Excitation Light]

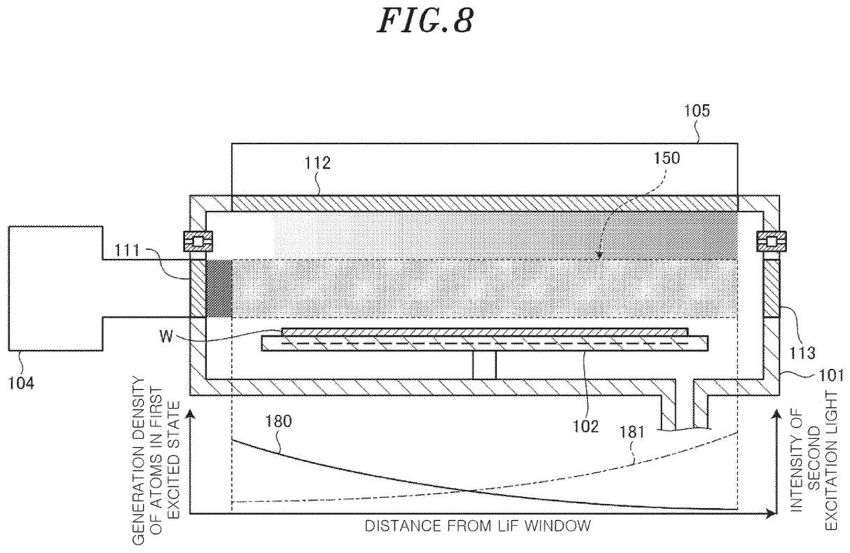

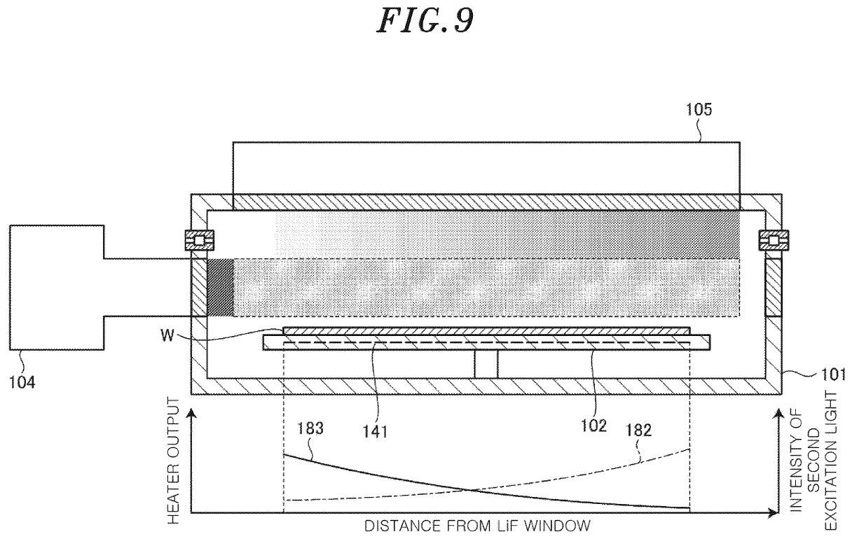

Next, the relationship between the distance from the irradiation window 111 (LiF window), the generation density of atoms in the first excited state, and the intensity of the second excitation light will be described with reference to FIGS. 8 and 9. FIG. 8 shows an example of the relationship between the distance from the lithium fluoride window, the generation density of atoms in the first excited state, and the intensity of the second excitation light. As shown in FIG. 8, the first excitation light emitted from the irradiation window 111 is absorbed by the noble gas in the processing chamber 101, and its intensity (light amount) decreases as the distance from the irradiation window 111 increases. In other words, as shown in graph 180, in the processing chamber 101, the generation density of atoms of the noble gas in the first excited state decreases as the distance from the irradiation window 111 (LiF window) increases. In contrast, as shown in graph 181, the intensity of the second excitation light is controlled to increase as the distance from the irradiation window 111 (LiF window) increases. In other words, the intensity of the second excitation light is controlled to form gradation in which the intensity increases as the distance from the irradiation window 111 (LiF window) increases. In other words, in the present embodiment, the spatial distribution of the second excitation light is adjusted to control the excitation efficiency from the first excited state to the second excited state. In other words, the gradation in the intensity of the second excitation light is controlled such that the emission during the transition of the noble gas from the second excited state to metastable excited atoms becomes uniform in the horizontal plane of the processing chamber 101 (particularly in the plane of the region where the substrate W is located). Accordingly, the metastable excited atoms can be generated uniformly in the region 150 above the substrate W to be processed.

FIG. 9 shows an example of the relationship between the distance from the lithium fluoride window, the heater output, and the intensity of the second excitation light. As shown in graph 182 of FIG. 9, the intensity of the second excitation light is controlled to increase as the distance from the irradiation window 111 (LIF window) increases. The substrate W is heated non-uniformly by the second excitation light, which is mainly infrared light, depending on the intensity distribution of the second excitation light. Therefore, in order to achieve a uniform temperature across the substrate W, as shown in graph 183, the output of the heater 141 is controlled to decrease as the distance from the irradiation window 111 (LIF window) increases. In other words, the output of the heater 141 is controlled such that gradation is formed which is reversed with respect to the gradation in the intensity of the second excitation light. In other words, the temperature of the placing table 102 is controlled to form gradation in the output of the heater 141 so as to be reversed with respect to the gradation in the intensity of the second excitation light. Accordingly, the temperature changes in the substrate W caused by the second excitation light can be corrected. Further, the intensity distribution of the second excitation light may be adjusted to compensate for the process non-uniformity caused by uneven flow of the noble gas and the processing gas in the processing chamber 101.

### [Substrate Processing Method]

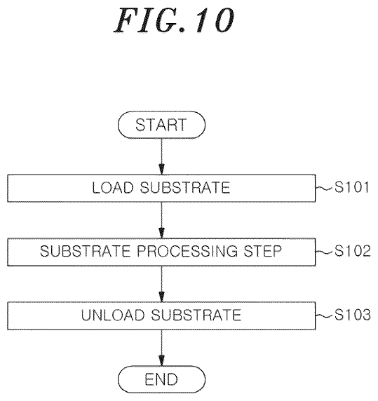

Next, a substrate processing method according to the first embodiment will be described. FIG. 10 is a flowchart illustrating an example of substrate processing according to the first embodiment.

The controller 11 controls a gate valve (not shown) to open a loading/unloading port (not shown). When the loading/unloading port is opened, the substrate W is loaded into the processing space 10s of the processing chamber 101 through the loading/unloading port and placed on the placing table 102. In other words, the controller 11 controls the substrate processing apparatus 1 to load the substrate W into the processing chamber 101 (step S101). Further, the controller 11 may be a control device for the entire substrate processing system (not shown) including the substrate processing apparatus 1 and a transfer device in a transfer chamber (not shown) adjacent to the processing chamber 101. The controller 11 controls the gate valve to close the loading/unloading port. Step S101 is an example of a process for loading and preparing the substrate W into the processing chamber 101.

The controller 11 controls the exhaust device 132 connected to the exhaust line 131 to reduce the pressure in the processing chamber 101 to a predetermined pressure. The controller 11 controls the gas supply part 123 to supply noble gas and a processing gas to the processing chamber 101 through the plurality of injection ports of the shower ring 121. The controller 11 controls the first excitation light source 104 to irradiate the processing chamber 101 with the first excitation light, thereby exciting the noble gas to the first excited state. Further, the controller 11 controls the second excitation light source 105 to irradiate the processing chamber 101 with the second excitation light, thereby allowing the noble gas to make the transition from the first excited state to the second excited state. The noble gas that has become metastable excited atoms through spontaneous emission from the second excited state is used to dissociate the processing gas, thereby processing the substrate W. The controller 11 measures the emission intensity distribution in the processing chamber 101 based on the emission data detected by the emission sensor 106 during the transition from the second excited state to metastable excited atoms. Based on the measured emission intensity distribution, the controller 11 performs feedback control on the second excitation light source 105 to ensure uniform emission across the horizontal plane, for example. In other words, the controller 11 controls the first excitation light source 104 and the second excitation light source 105 to perform a substrate processing step (step S102) in which the substrate W is processed using radicals generated from the processing gas by the noble gas that has become metastable excited atoms.

When the substrate processing step is completed, the controller 11 stops the first excitation light and the second excitation light to stop the generation of metastable excited atoms. Further, the controller 11 controls the gate valve to open the loading/unloading port. The controller 11 controls the substrate processing apparatus 1 such that the substrate W is lifted by causing substrate support pins (not shown) to protrude from the top surface of the placing table 102. When the loading/unloading port is opened, the substrate W is unloaded from the processing chamber 101 by an arm of the transfer chamber (not shown) through the loading/unloading port. In other words, the controller 11 controls the substrate processing apparatus 1 such that the substrate Wis unloaded from the processing chamber 101 (step S103). In this manner, the controller 11 controls the first excitation light source 104 and the second excitation light source 105 to uniformly generate metastable excited atoms. Further, in the present embodiment, radicals of the processing gas can be selectively generated, and ions and electrons are not generated, thereby suppressing damage to the substrate W due to ions and electrons. Further, by controlling the first excitation light source 104 and the second excitation light source 105, it is possible to more precisely control the distribution of metastable excited atoms, compared to the case of generating metastable excited atoms using plasma.

### Second Embodiment

In the first embodiment described above, the shower ring 121 was used to supply the noble gas and the processing gas. However, a substrate processing apparatus may also have a configuration in which a gas channel is provided in the irradiation window 112 to supply the noble gas and the processing gas in a shower-like manner from the upper surface of the substrate W. The present embodiment in this case will be described as the second embodiment. The substrate processing apparatus in the second embodiment is similar to the first embodiment except for the supply paths for the noble gas and the processing gas, so that the redundant description of the configurations and operations will be omitted.

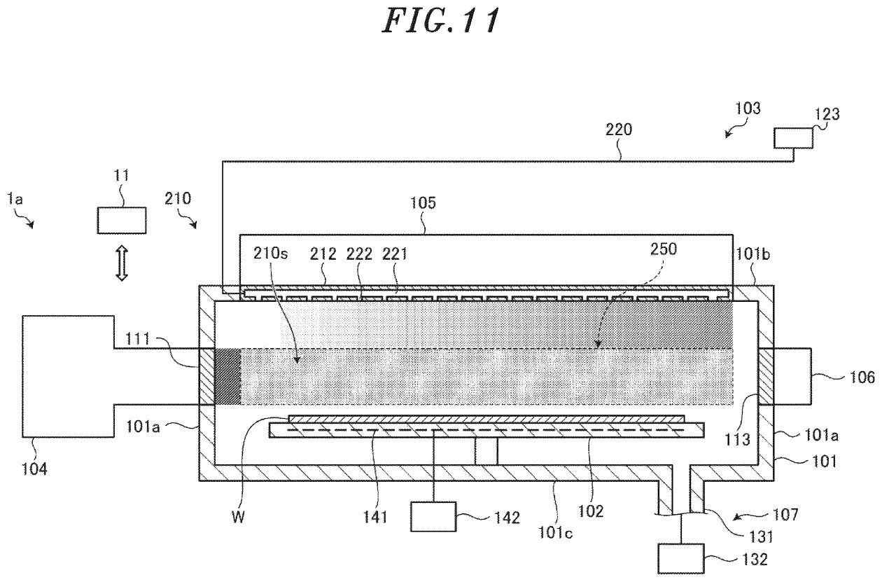

FIG. 11 is a schematic cross-sectional view showing an example of a configuration of the substrate processing apparatus in the second embodiment. As shown in FIG. 11, a substrate processing apparatus 1a of the second embodiment has a main body 210 instead of the main body 10 of the first embodiment. Further, the main body 210 has an irradiation window 212 instead of the irradiation window 112 of the first embodiment.

The irradiation window 212 has a gas channel 221 formed therein, and a plurality of injection holes 222 connected to the gas channel 221. One end of the gas channel 221 is connected to the gas supply part 123 through a line 220. The noble gas and the processing gas supplied to the gas channel 221 are supplied from the plurality of injection holes 222 to a processing space 210s to be uniformly distributed in the horizontal plane. In the processing space 210s, metastable excited atoms are generated in a region 250 irradiated with both the first excitation light and the second excitation light from the first excitation light source 104 and the second excitation light source 105, similarly to the region 150 of the first embodiment. In the region 250, the generated metastable excited atoms dissociate the processing gas, generating radicals. The substrate W is processed by the generated radicals. Thus, also in the substrate processing apparatus 1a of the second embodiment, metastable excited atoms can be uniformly generated by controlling the first excitation light source 104 and the second excitation light source 105.

In accordance with each embodiment, the processing apparatus (the substrate processing apparatuses 1 and 1a) includes the processing chamber 101, the gas supply source (the gas supply mechanism 103) configured to supply noble gas and a processing gas into the processing chamber 101, the first excitation light source 104 configured to irradiate the processing chamber 101 with the first excitation light to excite the noble gas to the first excited state, and the second excitation light source 105 configured to irradiate the processing chamber 101 with the second excitation light to irradiate the noble gas in the first excited state to the second excited state. As a result, metastable excited atoms can be generated uniformly.

Further, in accordance with each embodiment, the processing apparatus further includes the exhaust mechanism 107 configured to reduce the pressure in the processing chamber 101. As a result, metastable excited atoms can be generated uniformly in a depressurized atmosphere.

Further, in accordance with each embodiment, the processing apparatus further includes the emission sensor 106 configured to detect the emission generated when the noble gas in the second excited state becomes metastable excited atoms. As a result, the feedback control can be performed on the second excitation light source 105.

Further, in accordance with each embodiment, the first excitation light is irradiated from the side portion of the processing chamber 101, and the second excitation light is irradiated from the top of the processing chamber 101. As a result, damage to the substrate W due to the first excitation light, which is mainly vacuum ultraviolet light, can be reduced.

Further, in accordance with each embodiment, the second excitation light source 105 is provided in the processing chamber 101 such that the gradation in the intensity of the second excitation light is formed from the upstream side toward the downstream side of the first excitation light. As a result, metastable excited atoms can be uniformly generated.

Further, in accordance with each embodiment, the intensity of the second excitation light is set such that, when the upstream side and the downstream side of the first excitation light are compared, the intensity becomes weaker on the upstream side and stronger on the downstream side. As a result, metastable excited atoms can be generated uniformly.

Further, in accordance with each embodiment, the processing apparatus further includes the placing table 102 configured to incorporate the heater 141 in the processing chamber 101. The temperature of the placing table 102 is controlled such that gradation in an output of the heater 141 is formed so as to be reversed with respect to the gradation in the intensity of the second excitation light. As a result, the temperature changes in the substrate W due to the second excitation light, which is mainly infrared rays, can be corrected.

Further, in accordance with each embodiment, the processing apparatus further includes the emission sensor 106 configured to detect the emission during the transition of the noble gas from the second excited state to metastable excited atoms. The intensity gradation of the second excitation light is controlled such that the emission generated when the noble gas in the second excited state becomes metastable excited atoms becomes uniform across the horizontal plane of the processing chamber 101. As a result, metastable excited atoms can be generated uniformly.

Further, in accordance with each embodiment, the plurality of second excitation light sources (the light sources 160) are provided, and the intensity of the second excitation light is controlled for each of the plurality of second excitation light sources. As a result, the intensity of the second excitation light can be controlled more precisely.

Further, in accordance with each embodiment, the intensity gradation of the second excitation light is formed using the light control filter 171. As a result, various types of light sources can be used as the second excitation light source.

Further, in accordance with each embodiment, the light control filter is a liquid crystal filter. As a result, various types of light sources can be used as the second excitation light source, and the intensity of the second excitation light can be controlled more precisely.

Further, in accordance with each embodiment, the first excitation light is irradiated into the processing chamber 101 from the first excitation light source 104 through the lithium fluoride window (the irradiation window 111). As a result, vacuum ultraviolet light can be used as the first excitation light.

Further, in accordance with each embodiment, the second excitation light is irradiated into the processing chamber 101 from the second excitation light source 105 through an optical fiber and a quartz window (the irradiation window 112). As a result, the second excitation light source 105 can be located at a position other than the upper portion of the processing chamber 101.

Further, in accordance with each embodiment, the noble gas is argon gas. As a result, metastable excited argon atoms can be generated.

Further, in accordance with each embodiment, the processing gas is a silicon-containing gas. As a result, a silicon-containing film can be formed on the substrate W.

Further, in accordance with each embodiment, the processing gas is a halogen-containing gas. As a result, the inside of the processing chamber 101 can be cleaned. Further, the substrate W can be etched.

Further, in accordance with each embodiment, the processing method includes the steps of: supplying the noble gas and the processing gas into the processing chamber 101; exciting the noble gas to a first excited state by irradiating the processing chamber 101 with first excitation light; exciting the noble gas in the first excited state to a second excited state by irradiating the processing chamber 101 with second excitation light; and dissociating the processing gas with the noble gas that has become metastable excited atoms from the second excited state, thereby processing the substrate W. As a result, metastable excited atoms can be uniformly generated, and the substrate W can be processed with radicals of the processing gas dissociated by the metastable excited atoms.

Further, in accordance with each embodiment, the processing method further includes the step of detecting the emission generated when the noble gas in the second excited state becomes metastable excited atoms. As a result, the second excitation light can be controlled based on the emission during the transition of the noble gas to metastable excited atoms.

Further, in accordance with each embodiment, in the step of making the transition to the second excited state, the intensity of the second excitation light is controlled based on the emission detected in the step of detecting the emission. As a result, the feedback control of the intensity of the second excitation light can be performed based on the emission during the transition to the metastable excited atoms.

It should be noted that the above-described embodiments are illustrative in all respects and are not restrictive. The above-described embodiments may be omitted, replaced, or changed in various forms without departing from the scope of the appended claims and the gist thereof.

In the above-described embodiments, the processing chamber 101 has a substantially cylindrical shape, but the present disclosure is not limited thereto. For example, the processing chamber 101 may have any shape, such as a rectangular parallelepiped or the like. In other words, the distribution of the second excitation light can be controlled regardless of the shape of the processing chamber 101, so that metastable excited atoms can be uniformly generated.

The present disclosure may also include the following configurations.

(1) A processing apparatus comprising:
a processing chamber;
a gas supply source configured to supply noble gas and a processing gas into the processing chamber;
a first excitation light source configured to irradiate the processing chamber with first excitation light to excite the noble gas to a first excited state; and
a second excitation light source configured to irradiate the processing chamber with second excitation light to excite the noble gas in the first excited state to a second excited state.

(2) The processing apparatus of (1), further comprising:
an exhaust mechanism configured to reduce a pressure in the processing chamber.

(3) The processing apparatus (1) or (2), further comprising:
an emission sensor configured to detect emission generated when the noble gas in the second excited state becomes metastable excited atoms.

(4) The processing apparatus of any one of (1) to (3), wherein the first excitation light is irradiated from a side portion of the processing chamber, and the second excitation light is irradiated from the top of the processing chamber.

(5) The processing apparatus of (4), wherein the second excitation light source is provided in the processing chamber such that gradation in intensity of the second excitation light is formed from an upstream side toward a downstream side of the first excitation light.

(6) The processing apparatus of (5), wherein the intensity of the second excitation light is set such that, when the upstream side and the downstream side of the first excitation light are compared, the intensity becomes weaker on the upstream side and stronger on the downstream side.

(7) The processing apparatus of (5) or (6), further comprising:
a placing table configured to incorporate a heater in the processing chamber,
wherein the temperature of the placing table is controlled such that gradation in an output of the heater is formed so as to be reversed with respect to the gradation in the intensity of the second excitation light.

(8) The processing apparatus of any one of (5) to (7), further comprising:
an emission sensor configured to detect emission generated when the noble gas in the second excited state becomes the metastable excited atoms,
wherein the gradation in the intensity of the second excitation light is controlled such that the emission generated when the noble gas in the second excited state becomes the metastable excited atoms becomes uniform in a horizontal plane of the processing chamber.

(9) The processing apparatus of any one of (5) to (8), wherein a plurality of the second excitation light sources are provided, and the intensity of the second excitation light is controlled for each of the plurality of second excitation light sources.

(10) The processing apparatus of any one of (5) to (8), wherein the gradation in the intensity of the second excitation light is formed using a light control filter.

(11) The processing apparatus of (10), wherein the light control filter is a liquid crystal filter.

(12) The processing apparatus of any one of (1) to (11), wherein the first excitation light is irradiated into the processing chamber from the first excitation light source through a lithium fluoride window.

(13) The processing apparatus of any one of (1) to (12), wherein the second excitation light is irradiated into the processing chamber from the second excitation light source through an optical fiber and a quartz window.

(14) The processing apparatus of any one of (1) to (13), wherein the noble gas is argon gas.

(15) The processing apparatus of any one of (1) to (14), wherein the processing gas is a silicon-containing gas.

(16) The processing apparatus of any one of (1) to (15), wherein the processing gas is a halogen-containing gas.

(17) A processing method comprising:
supplying noble gas and a processing gas into a processing chamber;
exciting the noble gas to a first excited state by irradiating the processing chamber with first excitation light;
exciting the noble gas in the first excited state to a second excited state by irradiating the processing chamber with second excitation light; and
dissociating the processing gas with the noble gas that has become metastable excited atoms from the second excited state, thereby processing a substrate.

(18) The processing method of (17), further comprising:
detecting emission generated when the noble gas in the second excited state becomes the metastable excited atoms.

(19) The processing method of (18), wherein in said exciting the noble gas to the second excited state, the intensity of the second excitation light is controlled based on the emission detected in said detecting the emission.

1. A processing apparatus comprising:
a processing chamber;
a gas supply source configured to supply noble gas and a processing gas into the processing chamber;
a first excitation light source configured to irradiate the processing chamber with first excitation light to excite the noble gas to a first excited state; and
a second excitation light source configured to irradiate the processing chamber with second excitation light to excite the noble gas in the first excited state to a second excited state.

2. The processing apparatus of claim 1, further comprising:
an exhaust mechanism configured to reduce a pressure in the processing chamber.

3. The processing apparatus of claim 1, further comprising:
an emission sensor configured to detect emission generated when the noble gas in the second excited state becomes metastable excited atoms.

4. The processing apparatus of claim 1, wherein the first excitation light is irradiated from a side portion of the processing chamber, and
the second excitation light is irradiated from the top of the processing chamber.

5. The processing apparatus of claim 4, wherein the second excitation light source is provided in the processing chamber such that gradation in intensity of the second excitation light is formed from an upstream side toward a downstream side of the first excitation light.

6. The processing apparatus of claim 5, wherein the intensity of the second excitation light is set such that, when the upstream side and the downstream side of the first excitation light are compared, the intensity becomes weaker on the upstream side and stronger on the downstream side.

7. The processing apparatus of claim 5, further comprising:
a placing table configured to incorporate a heater in the processing chamber,
wherein the temperature of the placing table is controlled such that gradation in an output of the heater is formed so as to be reversed with respect to the gradation in the intensity of the second excitation light.

8. The processing apparatus of claim 5, further comprising:
an emission sensor configured to detect emission generated when the noble gas in the second excited state becomes the metastable excited atoms,
wherein the gradation in the intensity of the second excitation light is controlled such that the emission generated when the noble gas in the second excited state becomes the metastable excited atoms becomes uniform in a horizontal plane of the processing chamber.

9. The processing apparatus of claim 5, wherein a plurality of the second excitation light sources are provided, and the intensity of the second excitation light is controlled for each of the plurality of second excitation light sources.

10. The processing apparatus of claim 5, wherein the gradation in the intensity of the second excitation light is formed using a light control filter.

11. The processing apparatus of claim 10, wherein the light control filter is a liquid crystal filter.

12. The processing apparatus of claim 1, wherein the first excitation light is irradiated into the processing chamber from the first excitation light source through a lithium fluoride window.

13. The processing apparatus of claim 1, wherein the second excitation light is irradiated into the processing chamber from the second excitation light source through an optical fiber and a quartz window.

14. The processing apparatus of claim 1, wherein the noble gas is argon gas.

15. The processing apparatus of claim 1, wherein the processing gas is a silicon-containing gas.

16. The processing apparatus of claim 1, wherein the processing gas is a halogen-containing gas.

17. A processing method comprising:
supplying noble gas and a processing gas into a processing chamber;
exciting the noble gas to a first excited state by irradiating the processing chamber with first excitation light;
exciting the noble gas in the first excited state to a second excited state by irradiating the processing chamber with second excitation light; and
dissociating the processing gas with the noble gas that has become metastable excited atoms from the second excited state, thereby processing a substrate.

18. The processing method of claim 17, further comprising:
detecting emission generated when the noble gas in the second excited state becomes the metastable excited atoms.

19. The processing method of claim 18, wherein in said exciting the noble gas to the second excited state, the intensity of the second excitation light is controlled based on the emission detected in said detecting the emission.
